(19) United States
(12) Patent Application Publication
(10) Pub. No.: US 2007/0006368 A1
(43) Pub. Date: Jan. 11, 2007

(54) Disposable funnel for urine samples

(76) Inventor: Lori A. Key, West Hollywood, CA (US)

Correspondence Address: LITMAN LAW OFFICES, LTD, PO BOX 15035, CRYSTAL CITY STATION, ARLINGTON, VA, 22215, US

(21) Appl. No.: 11/174,623

(22) Filed: Jul. 6, 2005

(51) Int. Cl. A47K 11/00; A61B 5/00

(52) U.S. Cl. 4/144.2; 600/574; 4/144.1

(57) ABSTRACT

The disposable urine funnel is a urinary aid device that is placed upon a vaginal opening when the user is seated in order to guide urine into a urine sample cup. The disposable urine funnel is a bowl-shaped container that may be integrally connected to a tube, or the tube may be omitted entirely. The container has an upper lip that defines a mouth of the container. The mouth of the container has a first end and a second end, the first end being narrower from one side of the mouth to the other than the second end. The tube is optionally attached to the container and extends angularly downward from the container and in the direction of the first end of the mouth of the funnel, terminating in a discharge opening from which urine passes.

### BACKGROUND OF THE INVENTION

1. Field of the Invention

The present invention relates to urinary aids, and particularly to a disposable urine funnel.

2. Description of the Related Art

Many physical examinations or visits to a physician require a urine sample to be taken from the individual being examined. Generally, urine samples are taken by urinating into small paper cups provided by the physician. One of the numerous issues involved in obtaining these urine samples is that the urine tends to splatter in various directions, and urine is often dripped onto the hand of the individual holding the sample cup. This is both unsanitary and unpleasant for the individuals involved. Further, a problem with female patients is directing the urine flow into the small cup. Not only is it fairly difficult to direct the urine into such a small area, but it is necessary for accurate samples to be taken using a plentiful or sufficient amount of urine. If the user misses much of the sample cup, she may not provide enough urine to allow for an adequate sample.

In addition, young female children may find the prospect of obtaining a urine sample both difficult and distressing. These children may be quite uncomfortable attempting to obtain an adequate urine sample while trying to hold the cup to their vaginal opening. While parents may assist these children, the situation presented is still an awkward one for both parents and children alike.

Some urinary aids do exist that allow the urine stream to be directed. However, most of these devices are not disposable, and the user is required to wash and/or sanitize the device before it is reused. Most such urinary aids are designed to be used while the individual is standing, essentially providing a woman with a male-like extension to aim the device. These devices are quite unappealing and inelegant to numerous women and are generally not comfortable for use while sitting and urinating. Further, many do not provide an easy gripping means or cover the entire vaginal opening to eliminate splattering.

Accordingly, there is a need for a disposable urinary aid that allows a user to direct the flow of urine into a sample cup or collection device while sitting, that does not easily splatter, is easily gripped and is disposable once use is completed. Thus, a disposable urine funnel solving the aforementioned problems is desired.

### SUMMARY OF THE INVENTION

The disposable urine funnel is a urinary aid that allows a user to more easily collect urine samples. The disposable funnel is designed to be placed upon a vaginal opening when the user is seated in order to guide urine into a urine sample cup.

The disposable urine funnel is a bowl-shaped container that may be integrally connected to a tube or stem or that simply has an outlet, the tube being omitted entirely. The container has an upper lip that defines a mouth of the container. The mouth of the container has a first end and a second end, the first end being narrower from one side of the mouth to the other than the second end. The tube may be attached to the container and angularly extend downwardly from the container and in the direction of the first end of the mouth of the funnel, terminating in an open end. The tube is narrow and may extend under and past the lip of the first end of the mouth. The funnel is disposable and may be made from paper, plastic or any other type of disposable material.

The funnel is placed over the vaginal opening, the first end of the mouth of the container being positioned toward the front area of the vaginal opening. The user grips the tube and holds the funnel against the vaginal opening while the user is sitting. Urine is expelled into the funnel and flows through the mouth of the container and either out of the discharge opening or out of the tube, where the urine may be collected in a urine sample cup. This allows the user to place the urine funnel directly over the entire vaginal area when the user is seated, which provides increased coverage and less splatter when the funnel is being used to direct the flow of urine into the urine sample cup. The funnels may be stored in a disposable urine funnel dispenser.

These and other features of the present invention will become readily apparent upon further review of the following specification and drawings.

### BRIEF DESCRIPTION OF THE DRAWINGS

FIG. 1 is a perspective view of a disposable urine funnel according to a first embodiment of the present invention.

FIG. 2 is an elevational side view of the disposable urine funnel according to the first embodiment of the present invention.

FIG. 3 is a top view of the disposable urine funnel according to the first embodiment of the present invention.

FIG. 4 is an environmental, perspective view of a plurality of disposable urine funnels within a funnel dispenser.

FIG. 5 is an elevational side view of a disposable urine funnel according to a second embodiment of the present invention.

FIG. 6 is a top view of the disposable urine funnel of FIG. 5.

Similar reference characters denote corresponding features consistently throughout the attached drawings.

### DETAILED DESCRIPTION OF THE PREFERRED EMBODIMENT

The present invention is a disposable urine funnel used to collect urine samples. The disposable funnel, a first embodiment of which is designated generally as 10 in the drawings, is a funnel designed to be placed upon a vaginal opening when the user is seated in order to guide urine into a urine sample cup.

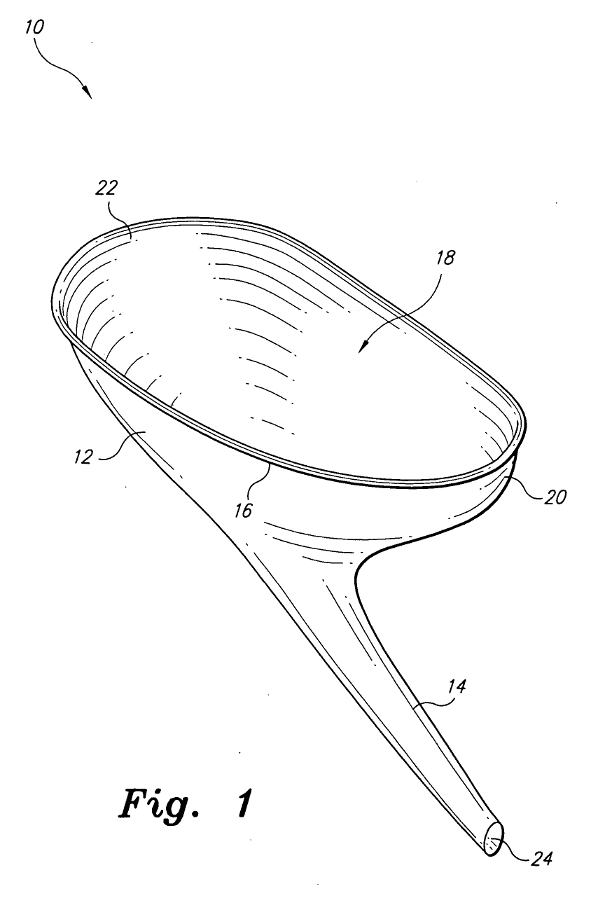

Referring first to FIG. 1, a perspective view of a first embodiment of the disposable funnel 10 is shown. The disposable urine funnel 10 is a bowl-shaped container 12 that is integrally connected to a tube 14. The bowl-shaped container 12 is generally egg-shaped and defines a reservoir. The funnel 10 is to be placed against the vaginal opening of a woman while she is using the bathroom facilities. The reservoir of the container 12 is adapted to receive urine. The container 12 has an upper lip 16 that defines a mouth 18 of the container 12. The upper lip 16 of the container 12 is meant to abut the user's vaginal opening.

The mouth 18 of the container 12 has a first end 20 and a second end 22 opposite the first end 20. The first end 20 of the container 12 is narrower than the second end 22 of the container 12. When the funnel 10 is being used, the first end 20 of the container 12 is positioned on the vaginal opening toward the front area of the vagina.

The funnel 10 may be made from any type of disposable material, including, but not limited to, paper or plastic.

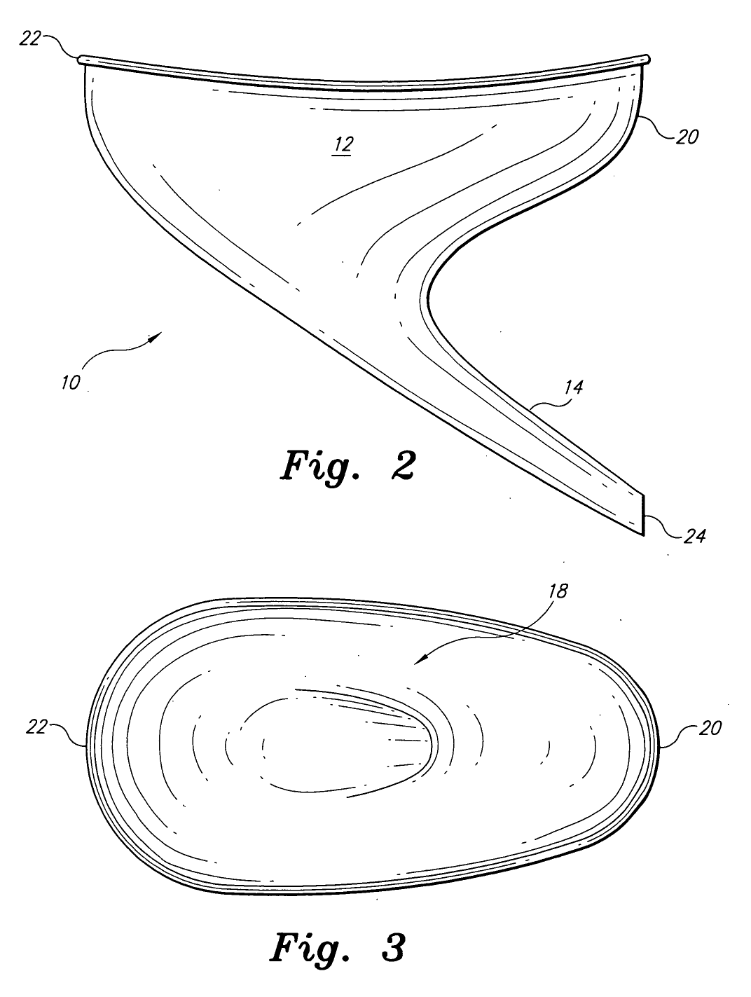

Turning now to FIG. 2, an elevational side view of the disposable funnel 10 is shown. The tube 14 is integrally connected to the container 12. The tube 14 extends angularly downward from the container 12 in the direction of the first end 20 of the mouth 18 of the container 12. The tube 14 terminates in a discharge opening 24. The discharge opening 24 of the tube 14 extends under the lip 16 of the first end 20 of the mouth 18 of the funnel 10. The tube 14 may optionally extend under the lip 16 and past the first end 20 of the mouth 18 of the funnel 10. The tube 14 is generally long and narrow and adapted to be gripped by a user while the individual is sitting on a toilet. Once urine is expelled into the container 12 through the mouth 18 of the container 12, the urine flows into the aperture within the tube 14 and out of the open end 24 of the tube 14.

FIG. 3 shows a top view of the disposable urine funnel 10. The first end 20 of the mouth 18 of the funnel 10 is narrower than the second end 22 of the mouth 18. The lateral distance across from one side of the mouth 18 to the opposite side at the first end 20 is less than that of the lateral distance across from one side of the mouth 18 to the opposite side at the second end 22. This allows the user to place the urine funnel 10 directly over the entire vaginal area when the user is seated, which provides increased coverage and less splatter when the funnel 10 is being used to direct the flow of urine into a urine sample cup.

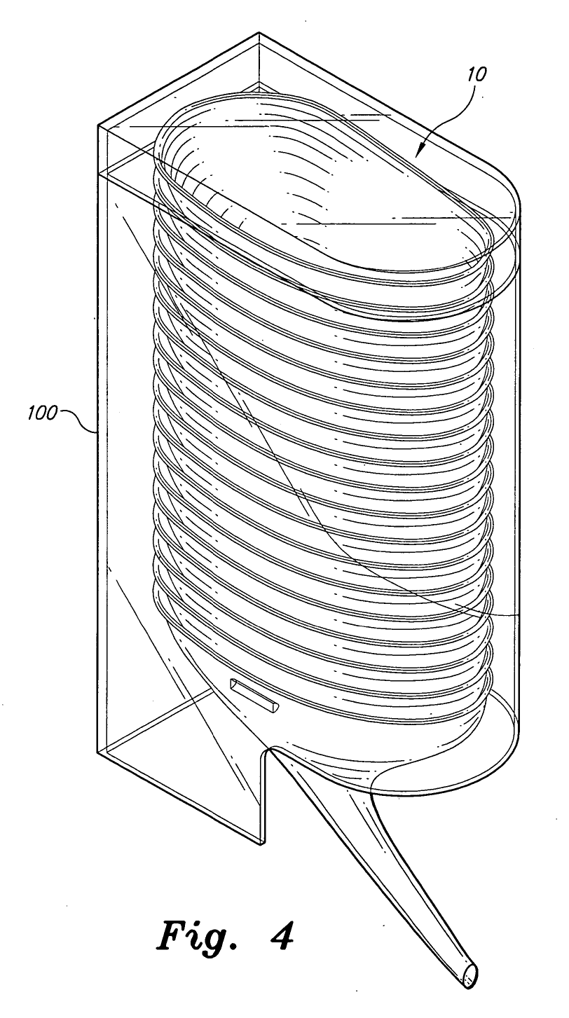

FIG. 4 shows an environmental, perspective view of a plurality of disposable urine funnels 10. As the urine funnels 10 are made from paper, plastic or any type of disposable material, the urine funnels 10 may be stacked one upon the other and held within a urine funnel dispenser 100. The funnels 10 may each by grasped by the tube 14 and be pulled out of the dispenser 100 in order to be used by the individual.

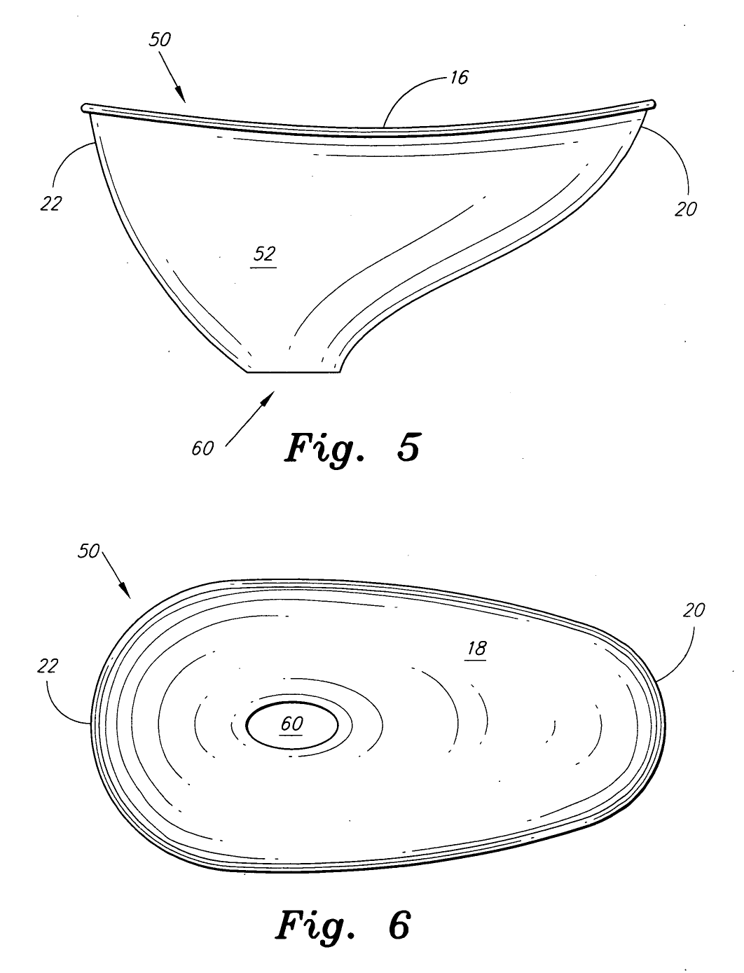

FIGS. 5 and 6 show a second embodiment of the urine funnel 50. The funnel 50 is a container 52 having an upper lip 16 that defines a mouth 18 (seen more clearly in FIG. 6) of the container 52. The bowl-shaped container 52 is generally egg-shaped and defines a reservoir. The mouth 18 of the container 52 has a first end 20 that is narrower than a second end 22. The lateral distance across from one side of the mouth 18 to the opposite side at the first end 20 is less than that of the lateral distance across from one side of the mouth 18 to the opposite side at the second end 22. The funnel 50 terminates in a discharge opening 60.

The discharge opening 60 of the urine funnel 50 is eccentrically located toward the second end 22 of the mouth 18 of the funnel 50. When the funnel 50 is placed against a woman's vaginal area, the first end 20 is placed toward the front of the vaginal area and the second end 22 is placed toward the back of the vaginal area. When the user urinates, the urine flows into the funnel 50, down the inside of the funnel 50 closest to the first end 20, and is expelled out through the discharge opening 60.

It is to be understood that the present invention is not limited to the embodiment described above, but encompasses any and all embodiments within the scope of the following claims.

I claim:

1. A disposable urine funnel, comprising:
a bowl-shaped container defining a reservoir, the container having a mouth with a first end and a second end, the first end being narrower than the second end and adapted to be positioned toward a front area of the vaginal opening; and
a tube integrally connected to the container and terminating in a discharge opening, the tube extending angularly downward in the direction of the first end of the container.

2. The disposable urine funnel according to claim 1, wherein the funnel is made from paper.

3. The disposable urine funnel according to claim 1, wherein the funnel is made from plastic.

4. The disposable urine funnel according to claim 1, further comprising an upper lip surrounding a perimeter of the mouth of the container.

5. The disposable funnel for urine samples according to claim 4, wherein the discharge opening of the tube extends under and past the lip of the container.

6. A disposable funnel for urine samples, comprising:
a bowl-shaped container defining a reservoir, the container having an upper lip adapted to cover a vaginal opening and defining a mouth of the container, the mouth having a first end and a second end, the first end being narrower than the second end and adapted to be positioned toward a front area of the vaginal opening; and
a tube integrally connected to the container and terminating in a discharge opening, the tube extending angularly downward in the direction of the first end of the container.

7. The disposable funnel for urine samples according to claim 6, wherein the funnel is made from paper.

8. The disposable funnel for urine samples according to claim 6, wherein the funnel is made from plastic.

9. The disposable funnel for urine samples according to claim 6, wherein the discharge opening of the tube extends under and past the lip of the container.

10. A disposable urine funnel, comprising a bowl-shaped container defining a reservoir, the container having a mouth defining a first end and a second end, the first end being narrower than the second end and adapted to be positioned toward a front area of the vaginal opening, the container having a lower portion and a discharge opening defined in the lower portion and eccentrically located toward the second end of the mouth.

11. The disposable urine funnel according to claim 10, wherein the funnel is made from paper.

12. The disposable urine funnel according to claim 10, wherein the funnel is made from plastic.

13. The disposable urine funnel according to claim 10, further comprising an upper lip surrounding a perimeter of the mouth of the container.
